(19) United States
(12) Patent Application Publication
(10) Pub. No.: US 2012/0065478 A1
(43) Pub. Date: Mar. 15, 2012

(54) INTERACTIVE BIOSENSING DEVICE AND METHOD THEREOF

(75) Inventors: BOR-SHYH LIN, CHANGHUA COUNTY (TW); CHIN-TENG LIN, HSINCHU COUNTY (TW); CHIH-FENG CHAO, TAICHUNG COUNTY (TW); SHAO-HANG HUNG, TAIPEI CITY (TW); I-JAN WANG, KAOHSIUNG CITY (TW)

(73) Assignee: NATIONAL CHIAO TUNG UNIVERSITY, HSINCHU CITY (TW)

(21) Appl. No.: 13/004,366

(22) Filed: Jan. 11, 2011

(30) Foreign Application Priority Data
Sep. 10, 2010 (TW) 99130606

(51) Int. Cl. A61B 5/00

(52) U.S. Cl. 600/301

(57) ABSTRACT

The present invention discloses an interactive biosensing device and a method thereof. The interactive biosensing device is arranged on a living body and comprises a physiological detection module detecting physiological signals of the living body; a physical sensation module triggering at least one physical device according to control instructions; a first wireless transmission module receiving control instructions or sends out physiological signals; a storage module storing physiological signals and control instructions; a video module capturing images of the living body; a control module processing physiological signals and control instructions and controlling operations of the video module; and a display unit presenting the images captured by the video module.

### BACKGROUND OF THE INVENTION

1. Field of the Invention

The present invention relates to an interactive biological technology, particularly to an interactive biosensing device and a method thereof.

2. Description of the Related Art

The current physiological sensing devices are usually for medical applications. For example, an electrocardiographic signal pickup device is arranged on a cardiopathic patient to detect electrocardiographic signals in realtime and transforms the analog signals into digital signals. A wireless transmission device may be installed in the electrocardiographic signal pickup device to transmit the electrocardiogram and related data to a hospital to implement a realtime-monitoring rescue system.

There are various physiological sensing devices. However, the conventional physiological sensing devices cannot interact. They cannot use the physiological signals of one side to control the actions of the other side. Besides, they are unlikely to undertake interaction between human beings and animals.

Accordingly, the present invention proposes an interactive biosensing device and a method thereof to overcome the abovementioned problems. The principles and embodiments thereof will be described in detail below.

### SUMMARY OF THE INVENTION

The primary objective of the present invention is to provide an interactive biosensing device and a method thereof, wherein two interactive biosensing devices are respectively arranged on two users to detect physiological signals of one user and send instructions or physical stimuli to the other user.

Another objective of the present invention is to provide an interactive biosensing device and a method thereof, which can be used to monitor or train pets.

To achieve the abovementioned objectives, the present invention proposes an interactive biosensing device, which is arranged on a living body, and which comprises a physiological detection module, a physical sensation module, a first wireless transmission module, a storage module, a video module, a control module and a display unit. The physiological detection module detects physiological signals of a living body. The physical sensation module triggers at least one physical device according to control instructions. The first wireless transmission module receives control instructions or sends out physiological signals. The storage module stores physiological signals and control instructions. The video module captures images of the living body. The control module processes physiological signals and control instructions, and controls operations of the video module. The display unit presents the images captured by the video module.

The present invention also proposes an interactive method for an interactive biosensing device, which comprises steps: using a physiological detection module of an interactive biosensing device to detect a physiological signal of a first living body; using a control module to transform the physiological signal into a control instruction and using a first wireless transmission module to transmit the control instruction to a second living body; and using an interactive biosensing device arranged on the second living body to receive the control instruction and using a physical sensation module to generate a physical stimulus or a reminder.

Below, the embodiments are described in detail to make easily understood the objectives, technical contents, characteristics and accomplishments of the present invention.

### BRIEF DESCRIPTION OF THE DRAWINGS

FIG. 1 is a diagram schematically showing the application of interactive biosensing devices according to a first embodiment of the present invention;

FIG. 2 is a block diagram schematically showing the architecture of an interactive biosensing device according to the first embodiment of the present invention;

FIG. 3 is a diagram schematically showing that the first embodiment of the present invention is applied to a pet;

FIG. 4 is a diagram schematically showing the application of interactive biosensing devices according to a second embodiment of the present invention;

FIG. 5 is a block diagram schematically showing the architecture of an interactive biosensing device according to the second embodiment of the present invention; and

FIG. 6 is a diagram schematically showing that the second embodiment of the present invention is applied to a pet.

### DETAILED DESCRIPTION OF THE INVENTION

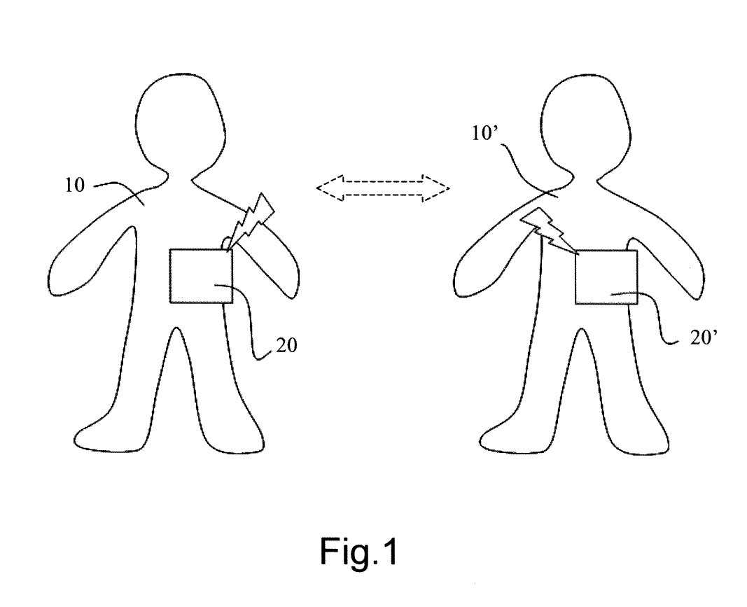

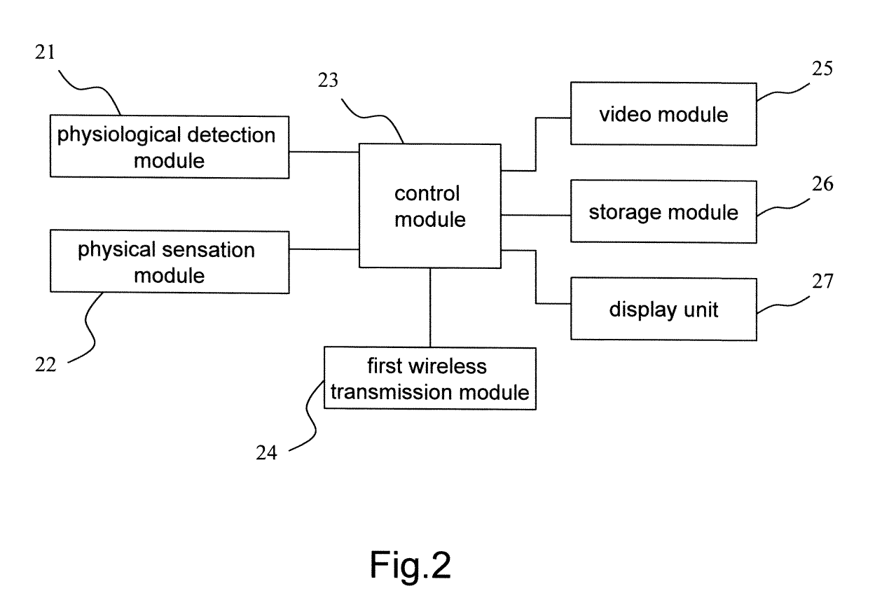

The present invention proposes an interactive biosensing device and a method thereof. Refer to FIG. 1 and FIG. 2. FIG. 1 is a diagram schematically showing the application of interactive biosensing devices according to a first embodiment of the present invention. FIG. 2 is a block diagram schematically showing the architecture of an interactive biosensing device according to the first embodiment of the present invention. The interactive biosensing device 20 of the present invention is installed on a living body, such as a human being or a pet. In FIG. 1, the interactive biosensing devices 20 and 20′ are respectively installed on a first user 10 and a second user 10′.

The interactive biosensing device 20 comprises a physiological detection module 21, a physical sensation module 22, a control module 23, a first wireless transmission module 24, a video module 25, a storage module 26, and a display unit 27. The physiological detection module 21 detects a physiological signal of the user 10. The physiological detection module 21 may be ECG (electrocardiography), EEG (electroencephalography), EMG (electromyography), EOG (electrooculography), a clinical thermometer, a tonometer or an oximeter, which is used to detect electrocardiographic signals, electroencephalographic signals, electromyographic signals, electrooculographic signals, body temperatures, blood pressures, or blood oxygen concentrations of the user 10. The physiological detection module 21 may be a vocal sensor, an accelerometer, a gyroscope, or a keyboard to detect the voices or actions of the user 10. The abovementioned sensors may be integrated to detect several types of signals. The physical sensation module 22 triggers at least one physical device according to a control instruction. The physical device may be a screen, a display, a light, a speaker, a vibrator, a heater, an electric stimulator, or a combination thereof. The first wireless transmission module 24 receives a control instruction and sends it to the physical sensation module 22, or transmits a physiological signal to the physiological detection module 21. The first wireless transmission module 24 is implemented with a wireless transmission technology of Bluetooth, ZigBee, 2G, 3G, 2.5G, 2.75G, 3G, Wi-Fi, or WiMAX. The video module 25 captures images of the user 10. The storage module 26 stores physiological signals, control instructions, and images captures by the video module 25. The control module 23 processes physiological signals and control instructions, determines whether the video module 25 begins to capture images, and controls operations of the video module 25. The display unit 27 presents images captured by the video module 25 and physiological data detected by the physiological detection module 21.

In the embodiment shown by FIG. 1 and FIG. 2, the interactive method of the present invention comprises steps: a physiological detection module 10 detecting a physiological signal of a first user 10; a control module 23 transforming the physiological signal into a control instruction, and a first wireless transmission module 24 transmitting the control instruction to a second user 10′; a first wireless transmission module 24 of an interactive biosensing device 20′ on the second user 10′ receiving the control instruction; a control module 23 of the interactive biosensing device 20′ on the second user 10′ transmitting the control instruction to a physical sensation module 22 of the interactive biosensing device 20′ on the second user 10′; the physical sensation module 22 generating a physical stimulus or a reminder to the second user 10′.

The interactive biosensing device 20 can use the video module 25 thereof to capture images of the first user 10 and use the first wireless transmission module 24 thereof to transmit the images to the interactive biosensing device 20′. The interactive biosensing device 20′ presents the images on the display unit 27 thereof, whereby the second user 10′ can watch the first user 10 and the surrounding thereof. In addition to physiological signals and control instructions, the images may be also stored in the storage module 26.

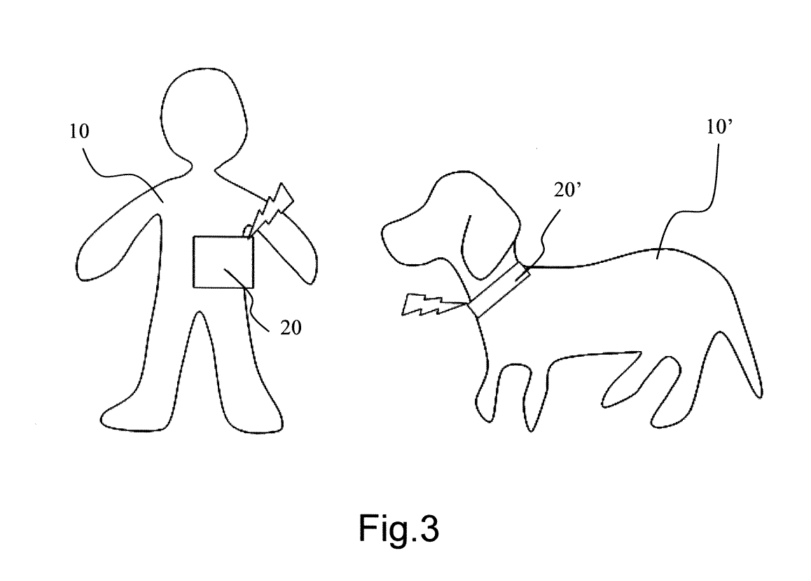

Refer to FIG. 3 a diagram schematically showing that the first embodiment is applied to a pet. The interactive biosensing device 20′ may be fabricated into a collar, which can be worn around the neck of a pet (the second user 10′). When the interactive biosensing device 20 receives a physiological signal of the master (the first user 10), it sends out a control signal. The interactive biosensing device 20′ of the pet receives the control signal and generates a physical stimulus or a reminder to instruct the pet to undertake some actions.

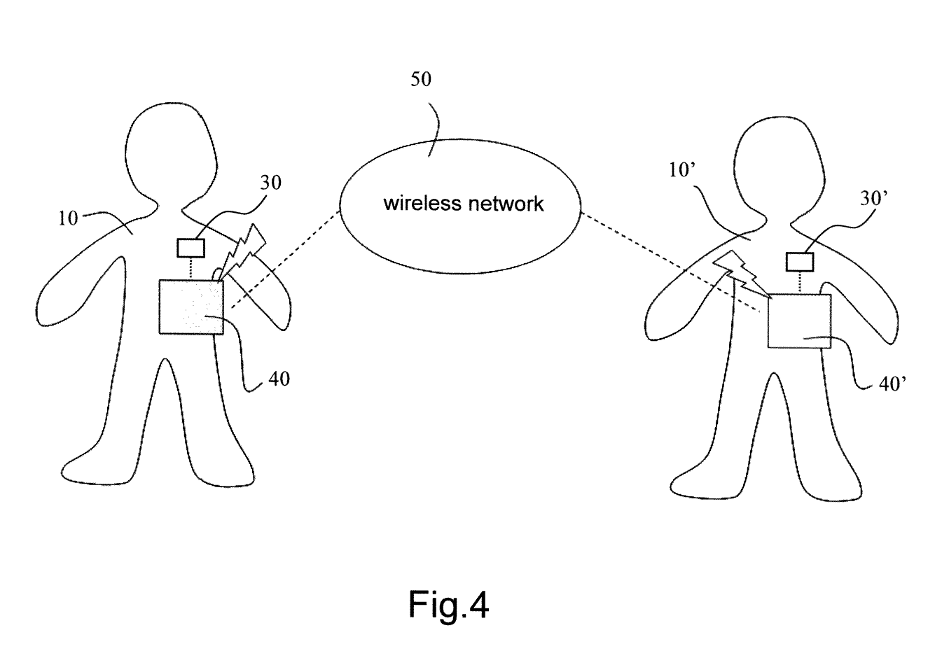

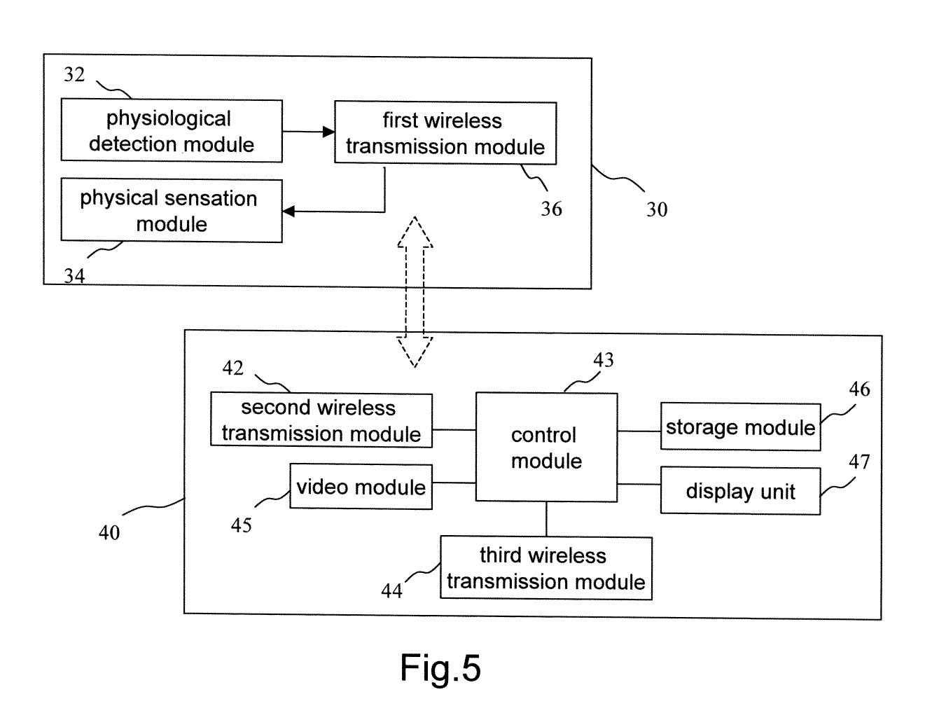

Refer to FIG. 4 and FIG. 5. FIG. 4 is a diagram schematically showing the application of interactive biosensing devices according to a second embodiment of the present invention. FIG. 5 is a block diagram schematically showing the architecture of an interactive biosensing device according to the second embodiment of the present invention. In this embodiment, an interactive biosensing device 30 is arranged on a living body 10, and there is further a host computer 40 also arranged on the living body 10. The interactive biosensing device 30 and the host computer 40 are wirelessly linked to each other. The living body 10 may be a human being or a pet. In the second embodiment shown in FIG. 4, the interactive biosensing device 30 and the host computer 40 are arranged on a first user 10; the interactive biosensing device 30′ and the host computer 40′ are arranged on a second user 10′. The host computer 40 is linked to the host computer 40′ via a wireless network 50. Both the host computers 40 and 40′ are portable devices, which may be in form of a mobile phone, PDA, a watch, a necklace, or a hand link.

In the second embodiment, the interactive biosensing device 30 comprises a physiological detection module 32, a physical sensation module 34, and a first wireless transmission module 36. The host computer 40 comprises a second wireless transmission module 42, a control module 43, a third wireless transmission module 44, a video module 45, a storage module 46, and a display unit 47. The physiological detection module 32 detects a physiological signal of the user 10. The physiological detection module 32 may be ECG (electrocardiography), EEG (electroencephalography), EMG (electromyography), EOG (electrooculography), a clinical thermometer, a tonometer or an oximeter, which is used to detect electrocardiographic signals, electroencephalographic signals, electromyographic signals, electrooculographic signals, body temperatures, blood pressures, or blood oxygen concentrations of the user 10. The physiological detection module 32 may be a vocal sensor, an accelerometer, a gyroscope or a keyboard to detect the voices or actions of the user 10. The abovementioned sensors may be integrated to detect several types of signals. The physical sensation module 34 triggers at least one physical device according to a control instruction. The physical device may be a screen, a display, a light, a speaker, a vibrator, a heater, an electric stimulator, or a combination thereof. The first wireless transmission module 36 and the second wireless transmission module 42 are implemented with a wireless transmission technology of Bluetooth, ZigBee, 2G, 3G, 2.5G, 2.75G, 3G, Wi-Fi, or WiMAX. The first wireless transmission module 36 transmits a physiological signal of the physiological detection module 32 to the second wireless transmission module 42. Alternatively, the second wireless transmission module 42 transmits a control instruction to the first wireless transmission module 36, and the first wireless transmission module 36 further sends the control instruction to the physical sensation module 34. The third wireless transmission module 44 is also implemented with a wireless transmission technology of Bluetooth, ZigBee, 2G, 3G, 2.5G, 2.75G, 3G, Wi-Fi, or WiMAX. The third wireless transmission modules 44 are used to perform intercommunication between the host computer 40 of the first user 10 and the host computer 40′ of the second user 10′. The video module 45 captures images of the user 10. The storage module 46 stores physiological signals, control instructions and images captures by the video module 45. The control module 43 processes physiological signals and control instructions, determines whether the video module 45 begins to capture images, and controls operations of the video module 45. The display unit 47 presents images captured by the video module 45 and physiological data detected by the physiological detection module 32.

In second embodiment shown by FIG. 4 and FIG. 5, the interactive method of the present invention comprises steps: a physiological detection module 32 detecting a physiological signal of a first user 10 and transmitting the physiological signal to a second wireless transmission module 42 of a host computer 40 via a first wireless transmission module 36; a control module 43 transforming the physiological signal into a control instruction; a third wireless transmission module 44 transmitting the control instruction to a second user 10′; a third wireless transmission module 44 of a host computer 40′ on the second user 10′ receiving the control instruction; the second wireless transmission module 42 of an interactive biosensing device 30′ on the second user 10′ transmitting the control instruction to the interactive biosensing device 30′ to drive a physical sensation module 34 to generate a physical stimulus or a reminder to the second user 10′.

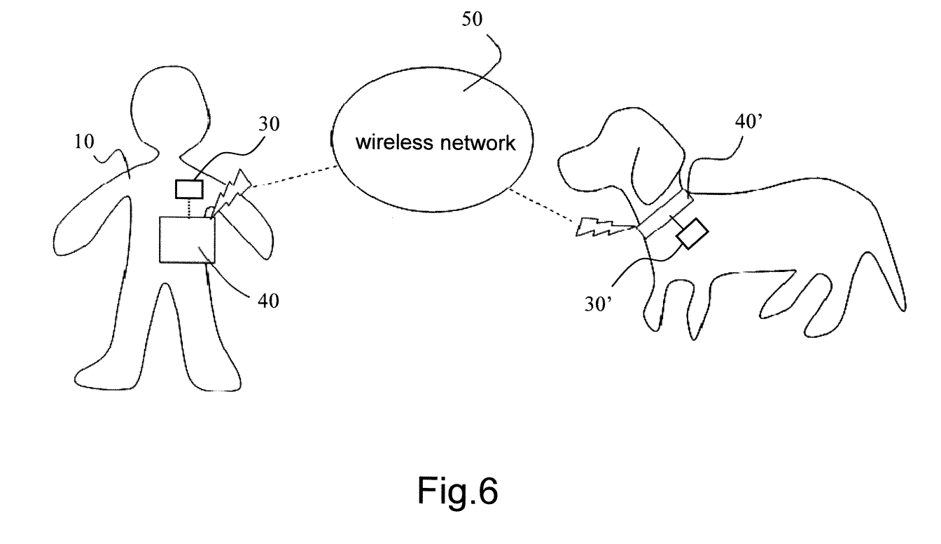

Refer to FIG. 6 a diagram schematically showing that the second embodiment is applied to a pet. The interactive biosensing device 30′ plus the host computer 40′ may be fabricated into a collar, which can be worn around the neck of a pet (the second user 10′). When the interactive biosensing device 30 receives a physiological signal of the master (the first user 10), it sends out a control signal. The interactive biosensing device 30′ of the pet receives the control signal, and the host computer 40′ generates a physical stimulus or a reminder to instruct the pet to undertake some actions. The video module 45 of the host computer 40′ can capture images of the pet, and the images are presented on the display unit 47 of the host computer 40 on the master.

In conclusion, the interactive biosensing device of the present invention not only can detect various physiological signals of a user but also can transmit the detection data to another user. The detection data may be presented in diversified forms. The user may be a human being or an animal. Thereby, the present invention enables one side to perceive the situation of the other side. Further, the present invention provides joyfulness of interaction for users. Furthermore, the present invention is not limited to only feeding back the detection data on a screen as the conventional technology but can present the detection data in diverse forms corresponding to various sensory organs.

The embodiments described above are only to exemplify the present invention but not to limit the scope of the present invention. Any equivalent modification or variation according to the spirit of the present invention is to be also included within the scope of the present invention.

What is claimed is:

1. An interactive biosensing device, which is arranged on a living body, comprising
a physiological detection module detecting a physiological signal of said living body;
a physical sensation module triggering at least one physical device according to a control instruction;
a first wireless transmission module receiving said control instruction or transmitting said physiological signal;
a storage module storing said physiological signal and said control instruction;
a video module capturing images of said living body;
a control module processing said physiological signal and said control instruction, and controlling operations of said video module; and
a display unit presenting images captured by said video module.

2. The interactive biosensing device according to claim 1, wherein said physiological detection module is ECG (electrocardiography), EEG (electroencephalography), EMG (electromyography), EOG (electrooculography), a clinical thermometer, a tonometer, an oximeter, a vocal sensor, an accelerometer, a gyroscope, a keyboard, or a combination thereof.

3. The interactive biosensing device according to claim 1, wherein said physical sensation module is a screen, a display, a light, a speaker, a vibrator, a heater, an electric stimulator, or a combination thereof.

4. The interactive biosensing device according to claim 1, wherein said first wireless transmission module is implemented with a wireless transmission technology of Bluetooth, ZigBee, 2G, 3G, 2.5G, 2.75G, 3G, Wi-Fi, or WiMAX.

5. The interactive biosensing device according to claim 1 further comprising a second wireless transmission module receiving a physiological signal from said first wireless transmission module, wherein said control module transforms said physiological signal into a control instruction, and wherein a third wireless transmission module transmits said control instruction to a far end.

6. The interactive biosensing device according to claim 5, wherein said third wireless transmission module receives a control instruction from a far end, and wherein said second wireless transmission module further transmits said control instruction to said first wireless transmission module.

7. The interactive biosensing device according to claim 5, wherein said storage module, said video module, said display unit, said control module, said second wireless transmission module and said third wireless transmission module are arranged in a host computer, and wherein said host computer is separated from said physiological detection module, said physical sensation module and said first wireless transmission module.

8. The interactive biosensing device according to claim 6, wherein said storage module, said video module, said display unit, said control module, said second wireless transmission module and said third wireless transmission module are arranged in a host computer, and wherein said host computer is separated from said physiological detection module, said physical sensation module and said first wireless transmission module.

9. The interactive biosensing device according to claim 5, wherein said second wireless transmission module and said third wireless transmission module are implemented with a wireless transmission technology of Bluetooth, ZigBee, 2G, 3G, 2.5G, 2.75G, 3G, Wi-Fi, or WiMAX.

10. An interactive method for interactive biosensing devices, which are respectively arranged on a first living body and a second living body, comprising steps:
using a physiological detection module of said interactive biosensing device to detect a physiological signal of said first living body;
using a control module to transform said physiological signal into a control instruction, and using a first wireless transmission module to transmit said control instruction to said second living body; and
said interactive biosensing device of said second living body receiving said control instruction and using a physical sensation module to generate a physical stimulus or a reminder.

11. The interactive method for interactive biosensing devices according to claim 10, wherein said interactive biosensing device further comprises a video module capturing images of said living body and a display unit presenting said images.

12. The interactive method for interactive biosensing devices according to claim 11, wherein said interactive biosensing device of said first living body uses said video module to capture images of said first living body and transmits said images to said interactive biosensing device of said second living body, whereby said second living body can watch said images of said first living body presented on said display unit.

13. The interactive method for interactive biosensing devices according to claim 10, wherein said first wireless transmission modules are respectively attached onto said first living body and said second living body.

14. The interactive method for interactive biosensing devices according to claim 10, wherein said interactive biosensing device further comprises a second wireless transmission device and a third wireless transmission device, and wherein said second wireless transmission device and said first wireless transmission device undertake short-distance communication therebetween, and wherein third wireless transmission device receives a control instruction from a far end or transmits a control instruction to a far end.

15. The interactive method for interactive biosensing devices according to claim 10, wherein said physiological detection module is ECG (electrocardiography), EEG (electroencephalography), EMG (electromyography), EOG (electrooculography), a clinical thermometer, a tonometer, an oximeter, a vocal sensor, an accelerometer, a gyroscope, a keyboard, or a combination thereof.

16. The interactive method for interactive biosensing devices according to claim 10, wherein said physical sensation module is a screen, a display, a light, a speaker, a vibrator, a heater, an electric stimulator, or a combination thereof.

17. The interactive method for interactive biosensing devices according to claim 10, wherein said first wireless transmission module is implemented with a wireless transmission technology of Bluetooth, ZigBee, 2G, 3G, 2.5G, 2.75G, 3G, Wi-Fi, or WiMAX.

18. The interactive method for interactive biosensing devices according to claim 14, wherein said second wireless transmission module and said third wireless transmission module are implemented with a wireless transmission technology of Bluetooth, ZigBee, 2G, 3G, 2.5G, 2.75G, 3G, Wi-Fi, or WiMAX.
